(19) United States
(12) Patent Application Publication
(10) Pub. No.: US 2022/0409112 A1
(43) Pub. Date: Dec. 29, 2022

(54) SYSTEM FOR MONITORING THE SLEEP AND/OR WELL-BEING OF A PASSENGER OF AN AIRCRAFT AND METHOD FOR IMPLEMENTING SUCH A SYSTEM

(71) Applicant: AIRBUS ATLANTIC, ROCHEFORT (FR)

(72) Inventors: Armand BORRAZ, PORTET-SUR-GARONNE (FR); Tanguy CAPITAINE, MARSILLY (FR); Julien DUCHESNE, SAINT LAURENT DE LA PRÉE (FR)

(21) Appl. No.: 17/801,248

(22) PCT Filed: Feb. 18, 2021
(86) PCT No.: PCT/EP2021/053957

§ 371 Date: Aug. 21, 2022

(30) Foreign Application Priority Data
Feb. 21, 2020 (FR) 2001732

(51) Int. Cl. A61B 5/16; A61B 5/00

(52) U.S. Cl. CPC A61B 5/165; A61B 5/6891; A61B 2503/12

(57) ABSTRACT

A system to manage the sleep and/or the well-being of a passenger/user of an aircraft includes a chair to be installed in a cabin of the aircraft and interaction devices to interact with the user. The system further includes a user terminal configured to receive data relating to characteristics of a user profile of the user, and a control/command module connected to the user terminal, interaction devices and a database storing information relating to characteristics of a flight of the aircraft. The command/control module being configured to pilot the interaction devices according to the data relating to characteristics of the user profile and characteristics of the flight of the aircraft.

### FIELD OF INVENTION

The present invention belongs to the field of equipment of an aircraft cabin and more particularly of the interactions between equipment of the cabin, in particular a chair, and a passenger. In particular, the present invention relates to a system for managing the sleep and/or the well-being of a passenger of an aircraft and a method for implementing such a system.

### BACKGROUND OF THE INVENTION

Crossing different time zones during the movement of an aircraft, in particular during commercial flights, generally induces a desynchronization of the biological clocks of the passengers of said aircraft, with respect to their life schedules. Indeed, the biological clocks of the passengers are set on the times of the departure country and not on those of the destination country. This desynchronization is commonly called “jet lag syndrome”.

For indication, at least ten days are necessary for the passengers of an aircraft for their biological clocks to adapt to the times of the destination country when the movement of the aircraft covers more than five time zones.

Depending on whether the direction of movement of the aircraft is directed westwards or eastwards, the jet lag syndrome is at the origin of difficulty falling asleep or causes early awakening. Hence, these sleep disorders generate sleep deprivation and consequently, a decrease in the cognitive performances and/or an increase in irritability in the concerned persons.

Moreover, because of recent developments enabling aircrafts to be lighter and more fuel efficient, we have been witnessing in recent years the apparition of aircrafts known to a person skilled in the art by the acronym ULR (standing for “Ultra Long Range”, meaning “ultra long range”), whose non-stop travel distance is very considerable.

Consequently, the passengers on these aircraft are likely to undergo a more significant jet lag syndrome.

In this context, there is a need to provide aircraft passengers with a solution allowing improving their management of the jet lag syndrome to minimize its impact, in particular the sleep disorders it causes.

### OBJECT AND SUMMARY OF THE INVENTION

An objective of the present invention is to overcome all or part of the drawbacks of the prior art, in particular those disclosed hereinbefore, by proposing a solution allowing managing the sleep and/or well-being level of a passenger of an aircraft, hereinafter called “user”, i.e., allowing favoring, preventing or slowing down drowsiness.

To this end, and according to a first aspect, the present invention proposes a system for managing the sleep and/or the well-being of a passenger of an aircraft, called “user”, characterized in that it comprises:
a chair intended to be installed in a cabin of an aircraft,
devices for interaction with the user,
user data acquisition means, configured to receive data relating to characteristics of a profile of a user, and
a control and command module connected:
a) to a database in which are stored information relating to characteristics of a flight of the aircraft in which the chair is intended to be installed,
b) to the interaction devices and
c) to the user data acquisition means, said control module being configured to pilot the interaction devices according to the data relating to characteristics of a profile of a user and characteristics of the flight of the aircraft, so as to favor falling asleep or keeping the user awake.

Such arrangements allow, after a travel on board an aircraft involving crossing different time zones, promoting the synchronization of the biological clocks of the passengers with the times of the destination country.

Hence, sleep disorders related to jet lag syndrome are avoided.

Advantageously, the sleep and/or well-being management system according to the present invention could be fitted on any existing aircraft chair.

In particular embodiments, the invention may further include one or more of the following features, considered separately or according to any technically-feasible combination.

In particular embodiments, the interaction devices comprise at least one of the following elements:
a temperature regulation device integrated into the chair,
a light device adapted to emit a light signal whose wavelength is variable between 400 nm and 700 nm,
a library of contextual messages stored in a database connected to a user terminal,
a sound environment generator,
at least one actuator cooperating with the chair so as to modify the position of said chair.

Advantageously, the temperature regulation device integrated into the chair allows controlling the temperature very accurately the closest to the passenger according to his feelings, which increases his comfort. The library of contextual messages stored in a database connected to a user terminal allows providing the passenger with information, in particular, the visualization of the customized program of successive tasks to be carried out over a given period. Also, the program may, during predefined time slots, provide for phases of falling asleep, sleeping, waking up, and/or rest and/or relaxation, neutral phase, etc. This allows him to increase his well-being and limiting the effects due to jet lag.

In particular embodiments, the user data acquisition means include devices for measuring the cardiac activity of the user and/or devices for measuring the respiratory rate.

In particular embodiments, the user data acquisition means include devices for measuring the physical activity.

In particular embodiments, the devices for measuring the cardiac activity and/or the devices for measuring the respiratory rate, and the devices for measuring the physical activity are derived from the same sensor integrated in the chair.

In particular embodiments, the control and command module is configured to:
determine a value representative of the nervous tension level and/or the drowsiness level of a user on the basis of the data relating to characteristics of the profile of the user,
establish a customized program for the user composed of successive tasks to be carried out over a given period, for example during the flight, according to information relating to the characteristics of a flight of the aircraft in which the chair is intended to be installed,
pilot the interaction devices according to said determined value and the customized program.

It should be noted that the considered flight is the flight that the user is intended to take as a passenger.

Furthermore, the piloting (or not) of the interaction devices is carried out at regular time intervals, according to the forecasts of the program and the nervous tension condition and/or the drowsiness level of the user at each of these time intervals.

In particular embodiments, the user data acquisition means comprise a user terminal adapted to receive contextual data of the user, the control and command module being configured to pilot the interaction devices according to these contextual data of the user.

According to a second aspect, the present invention relates to a method for managing the sleep and/or the well-being of a passenger, referred to as “user”, of an aircraft implementing a system as described before.

The method for managing the sleep and/or the well-being of a passenger of an aircraft, includes:
a step of acquiring data relating to a user profile and data relating to characteristics of a flight of the aircraft in which the chair intended to be occupied by said user is installed,
a step of piloting at least one device for interaction with the user according to the data relating to the user profile and the characteristics of the flight of the aircraft.

More specifically, the flight characteristics of the aircraft enable the establishment of a customized program of tasks to be carried out by the user, the program defining the steps of piloting the interaction devices. The data relating to the user profile will allow an update, in other words an evolution, of the program in real-time to the extent that the piloting steps will be confirmed or modified according to said data relating to the user profile.

In particular implementations, the step of acquiring data relating to a user profile, the nervous tension level and/or the drowsiness level of the user is determined on the basis of the data relating to characteristics of the profile of the user emitted by devices for measuring the cardiac activity of the user and/or devices for measuring the respiratory rate.

In particular implementations, the characteristic data of the flight of the aircraft include data relating to the scheduled take-off time of the aircraft with respect to the time zone of the take-off location, to the flight time of the aircraft and to the time of the destination location at the end of the flight, the piloting step not being implemented if the duration of the flight is less than a predefined value and/or if the value of the time of the destination location at the end of the flight is within a range of predefined values.

In particular implementations, the piloting step includes a conditioning step to favor keeping the user awake or a conditioning step to favor falling asleep according to the data relating to the user profile and the characteristics of the flight of the aircraft. In particular implementations, in the conditioning step to favor keeping the user awake, the control and command means pilot actuators so as to conform the chair in a seated position, and/or pilot a light device so that the light source emits a blue light.

In particular implementations, in the conditioning step to favor falling asleep, the control and command means pilot actuators so as to conform the chair in an elongated position, and/or pilot a light device so that the light source emits a red light, and/or pilot a temperature regulation device to increase the temperature of the chair, and/or pilot a sound environment generator.

In particular implementations, if the determined nervous tension level of the user is high, the piloting step implements as a priority a relaxation step in which the control and control module pilots actuators so as to conform the chair in a so-called relaxation position, and/or pilot the light device so that the light source emits a red light, and/or pilot a sound environment generator.

### BRIEF DESCRIPTION OF THE FIGURES

The invention will be better understood upon reading the following description, given as a non-limiting example, and made with reference to the figures which represent:

FIG. 1 schematically represents a system for managing the sleep and/or the well-being of a passenger of an aircraft according to the present invention, and

FIG. 2 is a flowchart of a method for managing the sleep and/or the well-being of a passenger of an aircraft according to another aspect of the present invention.

In these figures, identical references from one figure to another refer to identical or similar elements. For clarity, the represented elements are not necessarily to the same scale, unless stated otherwise.

It should be noted, as of now, that the figures are not to scale.

### DETAILED DESCRIPTION OF AN EMBODIMENT OF THE INVENTION

This description is given on a non-limiting basis, each feature of an embodiment may advantageously be combined with any other feature of any other embodiment.

As indicated before, the present invention relates to a system 10 for managing the sleep and/or the well-being of a passenger of an aircraft with respect to a predefined flight of said aircraft. The term “flight” herein refers to a route, in particular commercial, of the aircraft. The passenger is hereinafter referred to as the “user”.

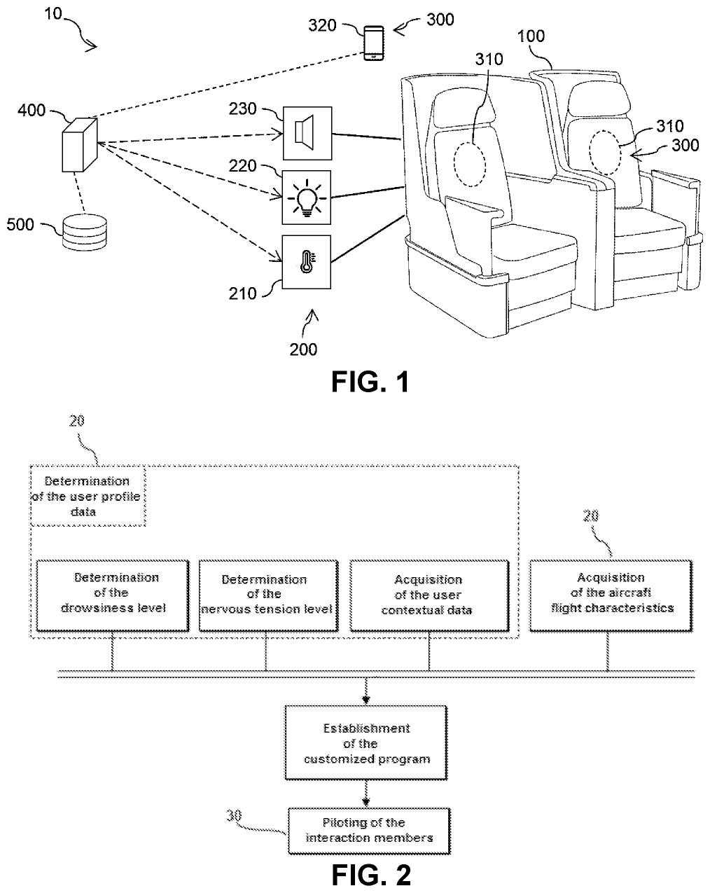

As represented by FIG. 1, the management system 10 comprises at least one chair 100 intended to be installed in a cabin of the aircraft, said chair 100 being attached to a user, devices 200 for interaction with said user, user data acquisition means 300.

The control and command module 400 is connected, on the one hand, to a database 500 in which are stored information relating to the characteristics of the flight of the aircraft in which the chair 100 is installed, and, on the other hand, to the interaction devices 200.

As described in more detail hereinafter, the control module is configured to pilot the interaction devices 200 according to the data relating to characteristics of a profile of a user and characteristics of the flight of the aircraft in order to enable the user to better manage his sleep and/or his well-being in order to reduce or avoid the effects of the jet lag syndrome.

Advantageously, the control and command module 400 includes calculation means, such as a processor adapted to implement one or more dedicated software.

The chair 100 is a chair conventional by its shape and its dimensions to the extent that it comprises a backrest, a seating area, armrests.

The devices 200 for interaction with the user may comprise equipment integrated into the chair 100, or possibly remote from said chair 100, such as a temperature regulation device 210, a light device 220, and/or one or more actuators arranged so as to be able to modify the position of the chair 100, and/or a sound environment generator 230.

More particularly, the thermal regulation device may be adapted to preferably heat or cool the user directly.

To this end, the thermal regulation device may comprise in particular a heating mat, and/or one or more Pelletier effect modules, integrated for example in the backrest and/or in the seating area of the chair 100.

For example, the light device 220 is integrated into the chair 100 at an upper portion of the backrest, for example intended to form a support for the head of the user, called “headrest”, and/or at the armrests. It could be considered that the luminous device 220 is integrated in any appropriate location of the chair 100, the determination of which is within the reach of a person skilled in the art.

Advantageously, the light device 220 is adapted to emit a light signal whose wavelength is variable between 400 nm and 700 nm.

To this end, the light device 220 preferably comprises several light sources, such as light-emitting diodes, respectively adapted to emit light signals of different wavelengths. For example, the light device 220 includes three light sources whose emitted light signals are respectively comprised between 460 nm and 480 nm, 540 nm and 560 nm, and 570 nm and 690 nm.

Preferably, a first actuator is advantageously arranged so as to be able to tilt the backrest between a “berth” type position, in which the backrest is tilted in a horizontal position, and a seated position in which the backrest is tilted in a vertical position.

Still in a preferred embodiment of the invention, a second actuator is arranged in the chair 100 so as to be able to deploy or retract a leg rest articulated to the seating area of the chair 100.

Still advantageously, the interaction devices 200 may comprise a sound environment generator 230. For example, said sound environment generator 230 may include active noise reduction modules and/or loudspeakers adapted to broadcast a predetermined soundscape.

The active noise reduction modules and/or the loudspeakers are integrated into the chair 100, for example in its backrest, in the headrest, or in any other suitable location of the chair 100.

The devices 200 for interaction with the user may further comprise equipment external to the chair 100, such as a library of contextual messages stored in a database 500 linked to a user terminal 320, as described in more detail hereinafter.

As shown in FIG. 1, the control and command module 400 is also connected to the user data acquisition means 300.

The user data acquisition means 300 are configured to receive data relating to characteristics of a profile of a user.

More specifically, the user data acquisition means 300 may be adapted to gather data relating to physiological characteristics of the user, and may comprise to this end devices for measuring the cardiac activity of the user and/or devices for measuring the respiratory rate, and/or devices for measuring the physical activity, i.e., the movements, of the user.

The term “measuring devices” herein refer to one or more sensors.

Preferably, the devices for measuring the cardiac activity, the respiratory rate and the physical activity are formed by the same sensor 310, integrated in the backrest, in the armrests, in the headrest, or in any other suitable location of the chair 100.

In the embodiment of the invention represented in FIG. 1, said sensor 310 is arranged in the backrest of the chair 100 and is schematically represented by broken lines.

Advantageously, this sensor 310 may include a plate on which is deposited a layer of a fluid whose conductivity depends on the deformation.

Thus, by supplying electricity to the fluid, when a force is exerted on the plate so as to deform said fluid, the variations in intensity of the transmitted electrical signal are such as to allow determining the position of the application point of the force as well as its intensity.

In this manner, the heart rate and respiratory rate could be determined.

The devices for measuring the cardiac activity, in particular the heart rate and the cardiac amplitude, may be formed by other appropriate sensors, for example known as such by a person skilled in the art.

The analysis of the cardiac and/or respiratory activity of the user allows determining the variation in the heart rate and/or the respiratory rate over a given period of time, and by exploiting these data, determining the nervous tension level of a user.

In this text, the terms “nervous tension” are used to refer to the stress condition of the user.

In particular, by the intervention of predefined algorithms implemented by the executed software, the control and command module 400 is configured to determine a value representative of the nervous tension level of a user.

The control and command module 400 may be configured so as to determine values representative of three nervous tension levels: low, medium, high.

In particular, a value determined in response to a heart rate lower than a predefined value, for example seventy beats per minute, corresponds to a low nervous tension level; a value determined in response to a measured heart rate comprised within a range of predefined values, for example between seventy and one hundred beats per minute, corresponds to a medium nervous tension level; a value determined in response to a measured heart rate higher than a predefined value, for example one hundred beats per minute, corresponds to a high nervous tension level.

To detect the nervous tension level more accurately and more reliably, it is preferable to take into account the combination of heart rate, heart amplitude and heart rate variation data over a given period of time.

As described hereinafter in more detail, the control and command module 400 is also configured to pilot the interaction devices 200 according to the determined value representative of three nervous tension levels, so as to tend to reduce the nervous tension level of said user.

More specifically, the control and command module 400 is configured to:
if the value corresponds to a low nervous tension level of the user, transmit no instructions to the interaction devices 200;
if the value corresponds to a medium nervous tension level of the user, pilot the actuators so as to conform the chair 100 in a so-called relaxation position, and/or pilot the light device 220 so that the light source emits a light red, i.e., a light whose wavelength is comprised between 570 nm and 690 nm. The relaxation position corresponds to a position in which the seating area of the chair 100 is tilted, for example by five to ten degrees, with respect to the horizontal, and the backrest of the chair 100 is tilted, for example by twenty to forty degrees, with respect to the vertical, the seating area and the backrest forming a right or obtuse angle.
if the value corresponds to a high nervous tension level of the user, in addition to piloting the actuators and/or the light device 220 as described when the nervous tension of the user is determined as medium, pilot the sound environment generator 230.

Advantageously, the data gathered by the measuring devices described hereinbefore, and more particularly the physical activity measuring devices, possibly in combination with the cardiac activity measuring devices and the respiratory rate measuring devices, additionally or alternatively, allow determining the drowsiness level of a user thanks to the data gathered by the user data acquisition means 300. In this text, the terms “drowsiness level” generally refer to the sleep phases, such as awakening, sleep, light sleep, deep sleep and paradoxical sleep.

In particular, by the intervention of predefined algorithms implemented by the executed software, the control and command module 400 is configured to determine a value representative of the drowsiness level of a user.

The physical activity measuring devices are also known as “actimeter” by a person skilled in the art and allow measuring the actimetry of the user, and thus gathering data representative of the physical activity of the user, in other words of the movements of the user, allowing analyzing the sleep-wake rhythm.

As described hereinafter in more detail, the control and command module 400 is also configured to pilot the interaction devices 200 according to the determined value, in order to influence the sleep and/or the well-being of the user, in particular so as to favor the drowsiness of the user or so as to tend to keep him awake in order to comply with a predetermined initial customized program of successive tasks to be carried out over a given period.

This program may be established by predefined algorithms implemented by software and executed by the control and command module 400, in particular according to the scheduled take-off time of the aircraft with respect to the time zone of the take-off location, the duration of the flight and the time of the destination location at the end of the flight.

Preferably, as schematically illustrated by the flowchart of FIG. 2, the decision of the control and command module to influence the drowsiness or the awakening of the user is a function, in addition to the values representative of the drowsiness level of the user, so that the customized program is progressively updated according to the physiological condition of the user.

In particular, if the control and command devices determine, through the exploitation of the data gathered in particular by the physical activity measuring devices, that the user is in a drowsy phase while the individual program has established an awake phase at this time point, said program could change to help the user fall asleep so as not to interfere with his sleep. More particularly, the evolution of the program may be such that the drowsiness of the user could be scheduled for a given duration. For example, according to said program, the control and command module 400 may be configured to pilot the interaction devices 200 so as to wake the user, for example thirty minutes after falling asleep.

As regards piloting of the devices for interaction with the user, the control and command module 400 is for example configured to:
if the objective established by the customized program is to keep the user awake, pilot the actuators so as to conform the chair 100 in a seated position, the backrest extending substantially vertically, i.e. perpendicular to the seating area, and/or pilot the light device 220 so that the light source emits a blue light, i.e. a light whose wavelength is comprised between 460 nm and 480 nm;
if the objective established by the customized program is to favor the drowsiness of the user, pilot the actuators so as to conform the chair 100 in an elongated position, the backrest being extended horizontally, i.e. in alignment with the seating area, and/or pilot the light device 220 so that the light source emits a red light, and/or pilot the temperature regulation device 210 so as to heat up the chair 100, for example up to at a rise of about eight degrees relative to an initial temperature of the chair 100, and/or pilot the sound environment generator 230.

Among the data relating to characteristics of a profile of a user, the user data acquisition means may advantageously be adapted to gather contextual data of the user 300, i.e., data that might inform on the environmental context in which the user is located before, during and after the flight.

Furthermore, these contextual data may for example be representative of the wishes belonging to the user with regards to the management of his drowsiness state, such as the wish to favor or not drowsiness thereof, to resist or not against the effects of the jet lag syndrome, the choice of the type of activity to be carried out during the flight, the purpose of the flight, for example if it is intended for a business or leisure trip.

The customized program may also be updated according to these contextual data.

To this end, the user data acquisition means 300 may comprise a user terminal 320, such as a smartphone commonly called “smartphone”, or a digital tablet, adapted to transmit and receive data, such as contextual data of the user, through a human-machine interface, for example a feedback touchscreen known per se to a person skilled in the art.

Alternatively, the term “user terminal” could refer to an electronic module integrated into the chair 100 also having a human-machine interface.

Taking into account the contextual data of the user enables the management system 10 to enable the user, for example through a dedicated interface on the screen of the user terminal 320, to view the customized program of successive tasks to be carried out over a given period. The program may provide, during predefined time slots, for phases of falling asleep, sleeping, waking up, and/or rest and/or relaxation, neutral phase, etc.

For example, the control and command module 400 may be configured so that, during such phases, it pilots the interaction devices 200 as follows:
during the drowsiness and sleeping phases, the actuators are piloted so as to conform the chair 100 in an elongated position, and/or the light device 220 is piloted so that the light source emits a red light;
during the awakening phase, the actuators are held in position so that the chair 100 keeps its lying position, possibly the actuators could be piloted so as to animate the chair 100 with slight movements repeated at regular intervals intended to wake the user, and/or the light device 220 is piloted so that the light source emits a white light progressively increasing in intensity, for example thirty minutes before the scheduled wake-up time until the scheduled wake-up time;
during the relaxation phase, the actuators are piloted so as to conform the chair 100 in the so-called relaxation position, and/or the light device 220 is piloted so that the light source emits a red light.

Alternatively or additionally, the management system 10, via the dedicated interface, could offer the user advices to follow according to the gathered contextual data. This feature may also be considered in the event of the determination of a given nervous tension level, for example a medium or high level, through the transmission of meditation/breathing advices.

The present invention also relates to a method for managing the sleep and/or the well-being implementing in particular the previously-described management system 10.

The method includes, as schematically represented in FIG. 2, a first step 20 of acquiring data relating to a user profile and data relating to the characteristics of a flight of the aircraft in which the chair 100 is installed.

In the step of acquiring 20 data relating to a user profile, the nervous tension level and/or the drowsiness level of the user is determined on the basis of the data relating to characteristics of the profile of the user emitted by the devices for measuring the cardiac activity of the user and/or by the devices for measuring the respiratory rate, and/or by the device for measuring the physical activity.

Following the data acquisition step 20, the method implements a step of piloting 30 at least one device 200 for interaction with the user as a function of the data relating to the user profile and the characteristics of the flight of the aircraft, for example following the above-described piloting instructions.

Advantageously, the piloting step 30 is not implemented if the value of the time difference between the takeoff location of the aircraft and the destination location at the end of the flight is less than a predefined duration, for example five hours.

More generally, it should be noted that the implementations and embodiments considered hereinabove have been described as non-limiting examples, and that other variants could consequently be considered.

1-14. (canceled)

15. A system to manage at least one of sleep and well-being of a passenger of an aircraft, the passenger being a user, comprising:
a chair intended to be installed in a cabin of the aircraft;
integration devices to interact with the user and comprising at least one of the following elements:
a temperature regulation device integrated into the chair;
a library of contextual messages stored in a database;
a user terminal connected to the database and configured to receive data relating to characteristics of a user profile of the user; and
a control and command module connected to:
the database additionally comprising information relating to characteristics of a flight of the aircraft in which the chair is intended to be installed;
the interaction devices; and
the user terminal;
wherein the control and command module being configured to pilot the interaction devices according to the data relating to characteristics of the user profile and characteristics of the flight of the aircraft.

16. The management system of claim 15, wherein the interaction devices comprise at least one of the following elements:
a light device configured to emit a light signal whose wavelength is variable between 400 nm and 700 nm;
a sound environment generator;
at least one actuator cooperating with the chair so as to modify a position of the chair.

17. The management system of claim 15, wherein the user terminal comprises at least one of devices to measure a cardiac activity of the user and devices to measure a respiratory rate of the user.

18. The management system of claim 17, wherein the user terminal comprises devices to measure a physical activity of the user.

19. The management system of claim 18, wherein the devices to measure the cardiac activity, the devices to measure the respiratory rate, and the devices to measure the physical activity are derived from a same sensor integrated in the chair.

20. The management system of claim 15, wherein the control and command module is configured to:
determine a value representative of at least one of a nervous tension level and a drowsiness level of the user in accordance with the data relating to characteristics of the user profile;
establish a customized program for the user composed of successive tasks to be carried out over a given period, according to information relating to the characteristics of the flight of the aircraft in which the chair is intended to be installed; and
pilot the interaction devices according to the value determined and the customized program.

21. The management system of claim 15, wherein the user terminal is configured to receive contextual data of the user, the contextual data relates an environmental context in which the user is located before, during and after the flight; and wherein the control and command module being configured to pilot the interaction devices according to the contextual data of the user.

22. A method for managing at least one of sleep and well-being of the passenger of the aircraft, the method implementing the management system of claim 15, comprising:
acquiring the data relating to the user profile and the data relating to characteristics of the flight of the aircraft in which the chair intended to be occupied by the user is installed; and
piloting at least one integration device according to the data relating to the user profile and the characteristics of the flight of the aircraft.

23. The management method of claim 22, wherein at least one of a nervous tension level and a drowsiness level of the user is determined in accordance with the data relating to characteristics of the user profile of the user emitted by devices to measure at least one of a cardiac activity, a respiratory rate and a physical activity of the user.

24. The management method of claim 22, wherein the data relating to the characteristics of the flight of the aircraft comprises data relating to a scheduled take-off time of the aircraft with respect to a time zone of a take-off location, a duration of flight of the aircraft and at a time of a destination location at an end of the flight.

25. The management method of claim 22, wherein the piloting said at least one integration device comprises conditioning to keep the user awake or to let the user fall asleep according to the data relating to the user profile and the characteristics of the flight of the aircraft.

26. The management method of claim 25, wherein in the conditioning to keep the user awake, the control and command module pilots at least one of the following: actuators to conform the chair in a seated position and a light device so that the light source emits a blue light.

27. The management method of claim 25, wherein in the conditioning step to let the user fall asleep, the control and command module pilots at least one of the following: actuators to conform the chair in an elongated position, a light device so that the light source emits a red light, a temperature regulation device to increase a temperature of the chair, and a sound environment generator.

28. The management method of claim 23, wherein in response to a determination that the nervous tension level of the user determined is higher than a predetermined level, the command and control module pilots at least one of the following to implement as a priority a relaxation step: actuators to conform the chair in a relaxation position, a light device so that the light source emits a red light, and a sound environment generator.
